(19) United States
(12) Patent Application Publication
(10) Pub. No.: US 2026/0251511 A1
(43) Pub. Date: Aug. 27, 2026

(54) METHOD AND APPARATUS FOR CAPACITIVE FORCE SENSING

(71) Applicant: VIDICOR INC, Laguna Beach, CA (US)

(72) Inventor: Alexander Holland, Laguna Beach, CA (US)

(21) Appl. No.: 19/546,196

(22) Filed: Feb. 20, 2026

Related U.S. Application Data
(60) Provisional application No. 63/761,953, filed on Feb. 22, 2025.
(60) Provisional application No. 63/979,830, filed on Feb. 10, 2026.
(60) Provisional application No. 63/982,764, filed on Feb. 13, 2026.

(51) Int. Cl. G01L 1/14

(52) U.S. Cl. CPC G01L 1/144; G01L 1/146

(57) ABSTRACT

Methods and apparatuses for force sensing using a thin and flexible capacitive transducer surface. The thin and flexible capacitive transducer can be a variety of sizes and shapes, allowing it to conform to non-flat surfaces with widely varying surface pressure contact patterns. The sensing system's capacitive pressure transducer can include multiple layers of flexible electrodes separated by compressible dielectric. When nonuniform surface pressure patterns are applied, the dielectric layers undergo nonuniform distortion and compression across their surfaces producing an overall change in capacitance that analog and digital signal processing methods convert to the total applied load. The transducer is in effect a pressure integrating surface that does not require rigid contact area anywhere.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the benefit and priority of U.S. Provisional Application No. 63/761,953 filed Feb. 22, 2025 and titled “FootSens: A Smart Shoe Platform,” of U.S. Provisional Application No. 63/979,830 filed Feb. 10, 2026 and titled “FootSens: A Smart Footwear Platform,” and of U.S. Provisional Application No. 63/982,764 filed Feb. 13, 2026 and titled “FootSens: A Smart Shoe Platform,” the entire content of each of which is hereby incorporated by reference and forms a part of this specification for all purposes.

### BACKGROUND

### Field

The present disclosure relates to force sensors or sensing systems. More specifically, it relates to methods and apparatus for measuring force using capacitive sensing techniques, and may include either local or remote digital signal processing to extract force data from measured digital signals.

### Description of the Related Art

Capacitive force sensors commonly detect or measure mechanical loads by measuring changes in capacitance resulting from deformation of a dielectric material due to displacement between conductive elements on either side of the dielectric. These elements are often thin, flat, parallel plates, where the plates can have almost any two-dimensional geometry. A rigid or flat contact boundary converts pressures or forces normal to its surface into a uniform pressure across the known area of its plate electrodes causing a uniform compression of the dielectric layer that reduces the separation between electrodes and thereby increases capacitance. The total force normal to the sensor's rigid boundary is linearly or nonlinearly proportional to the capacitance increase, depending on the respective linear or nonlinear elastic characteristics of the dielectric. A rigid or near-rigid boundary is necessary to maintain consistent performance and ensure repeatable response from different distributions of force or pressure patterns that may occur on the contact surface.

Conventional flexible capacitive sensors detect forces or pressures, but cannot measure force accurately without some rigid boundary assumptions or without calibration using a specific surface pressure distribution and are useful primarily for that distribution.

For example, CA 2352768 A1 relates to a flexible capacitive force transducer that provides “feedback on pressure or force”, but does not quantitatively measure the force in a standard unit such as Newtons or pounds. Similarly, US 2020/0064918 A1 pertains to capacitive flex sensors attached to gloves or bendable surfaces and provides information on movement. U.S. Pat. No. 7,395,717 B2 describes a (a) flexible capacitive sensor that measures pressure immediately above the sensor, as well as (b) sensor array techniques to locate pressure points.

Some conventional force sensors may employ a wider sensing boundary that is not completely rigid, but these sensors employ a more complex design geometry to concentrate the force or pressure patterns from a wider surface area onto a smaller rigid or flat area directly above the sensor. An example is shown by Muzaffar et al. in “Shoe Integrated, Force Sensor Design for Continuous Body Weight Monitoring” (Sensors, 2020), where layered consolidator structures embedded in an insole estimate body weight.

US Patent Publication 2018/0078176 A1 describes a capacitive sensor architecture employing a single flexible dielectric layer formed from an elastomer containing a plurality of gaps or voids. These gaps partition the dielectric into an array of elastomeric pillars that function as spaced spring members, over which substantially continuous top and bottom conductive electrodes are attached. The presence of the gaps permits the use of volume-incompressible elastomers, enabling one-dimensional compression to be governed primarily by the elastic response of the pillars rather than by the high bulk modulus of a nearly incompressible volume. As a result, only portions of the dielectric may be in contact with the conductive electrodes at any given time. In practice, the use of substantially volume-incompressible elastomers as capacitive dielectrics, such as rubbers, silicones, or polyurethanes, necessitates the introduction of spacings, voids, or geometric discontinuities to enable meaningful compressive deformation. Absent such gaps, compression would be dominated by the material bulk modulus, which is orders of magnitude larger than the elastic modulus, thereby severely limiting achievable strain. Consistent with this principle, Mannsfeld et al. reported pressure sensors employing rubber dielectrics structured as arrays of pyramidal unit cells, while Kwon et al. and Gerratt et al. utilized porous dielectric materials for pressure sensing applications.

### SUMMARY

The embodiments disclosed herein each have several aspects, of which no single one is solely responsible for the disclosure's desirable attributes. Without limiting the scope of this disclosure, the prominent features are briefly discussed herein. After considering this discussion, and particularly after reading the section entitled “Detailed Description,” one will understand how the features of the embodiments described herein provide advantages over existing systems, devices, and methods relating to force sensing.

In contrast to conventional designs, the capacitive force sensor according to the present disclosure in some embodiments operates without requiring a rigid contact boundary and can sense over a large flexible contact area, enabling greater mechanical flexibility and integration into soft or deformable structures, particularly for applications requiring thin, low-cost, or more conformable sensors, such as those used in wearable electronics, medical devices, smart insoles, robotic grippers or contacts, or other force or pressure sensing surfaces.

In an aspect, the present disclosure provides for a capacitive force sensing apparatus including: (a) a flexible capacitive transducer, the flexible capacitive transducer including electrodes separated by at least one deformable dielectric, the flexible capacitive transducer defining a pressure-integrating sensing surface; (b) a capacitance measurement circuit coupled to the electrodes and configured to measure an aggregate capacitance of the flexible capacitive transducer; and (c) one or more processors executing instructions to: (i) estimate the aggregate capacitance indicative of a spatially non-uniform pressure distribution applied across the pressure-integrating sensing surface, and (ii) determine, from the aggregate capacitance, a quantitative estimate of a total applied force, where: the quantitative estimate of total applied force is substantially invariant to a spatial distribution of the applied pressure over the sensing surface, and the capacitive force sensing apparatus is without a rigid backing plate, a rigid load-spreading structure, or a rigid boundary that enforces a uniform pressure field across a surface of the flexible capacitive transducer.

In some embodiments, the flexible capacitive transducer includes a plurality of flexible electrode layers and a plurality of deformable dielectric layers vertically interdigitated to form a multilayer electrode-dielectric stack defining a plurality of capacitors electrically connectable in parallel. In some further embodiments, an aggregate capacitance of the multilayer electrode-dielectric stack increases approximately with a square of a number of dielectric layers in the stack. In some further embodiments, the multilayer electrode-dielectric stack includes alternating signal electrode layers and reference electrode layers. In yet further embodiments, at least one reference electrode layer is held at a ground or guard potential, where the at least one reference electrode layer held at a ground or guard potential is configured to provide electromagnetic interference shielding. In some further embodiments, the multilayer electrode-dielectric stack includes at least three dielectric layers.

In some embodiments, at least one deformable dielectric exhibits an effective compressive stiffness that increases with applied pressure. In further embodiments, the effective compressive stiffness can follow or approximately follow

$$p = {E}_{0} \frac{δ}{1 - δ}$$

for pressure p and strain δ over an operating pressure range. In some further embodiments, the effective compressive stiffness increase enables an approximately proportional relationship between capacitance change and total applied force over a larger strain range than a constant-stiffness dielectric. In some further embodiments, the deformable dielectric includes a structured or composite dielectric configured to produce the effective stiffness increase with applied pressure.

In some embodiments, the flexible capacitive transducer includes a multilayer electrode-dielectric stack with vertically interdigitated electrode and dielectric layers forming a plurality of capacitors in parallel, and at least one dielectric layer of the multilayer electrode-dielectric stack exhibits an effective compressive stiffness that increases with applied pressure. In some embodiments, the one or more processors execute instructions to apply a calibrated mapping from measured capacitance to total force. In some further embodiments, the calibrated mapping is nonlinear and compensates for at least one of dielectric nonlinearity, environmental variation, or material aging. In some further embodiments, the calibrated mapping is trained using supervised learning with reference force values.

In some embodiments, the capacitance measurement circuit can apply a time-varying stimulus to the flexible capacitive transducer and estimates capacitance based on a response of the transducer. In some further embodiments, the time-varying stimulus includes a step waveform and capacitance is estimated from a step-response time constant. In some further embodiments, both the stimulus and a response signal are digitized and capacitance is estimated using an adaptive model. In some embodiments, the flexible capacitive transducer conforms to a non-planar surface in an unloaded state. In some embodiments, the flexible capacitive transducer is integrated into a wearable or deformable structure selected from an insole, shoe, slipper, sock, textile, rug, mat, or robotic contact surface.

In another aspect, the present disclosure provides for a flexible capacitive transducer including: (a) flexible electrode layers; and (b) at least one deformable dielectric separating opposing electrode layers; where the transducer is configured to produce an aggregate capacitance change representative of a total force applied under non-uniform pressure distributions without requiring a rigid backing structure.

In some embodiments, the transducer includes vertically interdigitated electrode and dielectric layers forming a plurality of capacitors in parallel. In some embodiments, the deformable dielectric exhibits an effective compressive stiffness that increases with applied pressure according to or approximately according to

$$p = {E}_{0} \frac{δ}{1 - δ}$$

In some embodiments, the transducer includes both vertically interdigitated electrode-dielectric layers and a pressure-dependent effective dielectric stiffness.

In another aspect, the techniques described herein relate to a method for sensing force including: (a) providing a flexible capacitive transducer including flexible electrodes separated by at least one deformable dielectric; (b) measuring a capacitance-dependent signal from the flexible capacitive transducer while a non-uniform pressure distribution is applied; (c) estimating capacitance from the capacitance-dependent signal; and (d) generating an estimate of a total applied force from the estimated capacitance, where the method is performed without constraining the flexible capacitive transducer to a rigid planar boundary.

### BRIEF DESCRIPTION OF THE DRAWINGS

The foregoing and other features of the present disclosure will become more fully apparent from the following description and appended claims, taken in conjunction with the accompanying drawings. In the following detailed description, reference is made to the accompanying drawings, which form a part hereof. Understanding that these drawings depict only several embodiments in accordance with the disclosure and are not to be considered limiting of its scope, the disclosure will be described with additional specificity and detail through use of the accompanying drawings. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, may be arranged, substituted, combined, and designed in a wide variety of different configurations, all of which are explicitly contemplated and made part of this disclosure.

FIG. 1 is a block diagram illustrating the sensor. The blocks are Digital Electronics (100), Analog & Conversion Electronics (200), and Flexible Capacitive Transducer (300) that may cover a significant area with no portion necessarily rigid in order to measure total force impinging on the transducer surface.

FIG. 2 is a view of a single layer for the Flexible Capacitive Transducer (300), illustrating conductive Top Electrode Layer (301) and conductive Bottom Electrode Layer (303) with nominal electrode separation via insulating Elastic Dielectric Layer (302) that results in a baseline capacitance.

FIG. 3 is a block diagram of the digital processing done by Digital Electronics (100). Digital Signal Generation (101) generates the input stimulus waveform. Digital Response Processing (102a) estimates total force that uses only the output digital response signal samples.

FIG. 4 is a more detailed block diagram for another embodiment of Digital Electronics (100). Digital Signal Generation (101) generates the input stimulus waveform. Digital Response Processing (102b) estimates total force by processing digital signal measurement samples of both the input stimulus and output response using an adaptive machine learning method.

FIG. 5 shows a 2-layer dielectric transducer design with interference shielding and a cross-section cut to show the inside layers that have 4 times increased sensitivity.

FIG. 6 illustrates an N-layer dielectric transducer design with interference shielding and a cross-section cut to show inside layers that N² (“N” squared) times increased sensitivity, where in this case N=6.

FIG. 7 shows an M×P two-dimensional array of N-Layer flexible transducers as shown in FIG. 6, where M, N, and P are integers greater than or equal to 1.

FIG. 8 shows an M×P two-dimensional array of N-Layer flexible transducers as shown in FIG. 6, where M, N, and P are integers greater than or equal to 1 and where the array is encoded as M outer conductor strips overlapped with P inner conductor strips.

FIG. 9 shows corresponding nonlinear signal processing for an M×P two-dimensional array of N-Layer flexible transducers.

FIG. 10 shows a force sensing computing system.

FIG. 11 shows a surface texture that provides a second order approximation to an ideal desired nonlinear stress/strain curve. The second order approximation provides an order of magnitude improved accuracy and sensitivity over a linear approximation.

### DETAILED DESCRIPTION OF SOME EMBODIMENTS

The following detailed description is directed to certain specific embodiments of systems, devices, and methods for force sensing. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments of the invention, examples of which are illustrated in the accompanying drawings, are set forth in detail below. Wherever possible, the same reference numbers are used throughout the drawings to refer to the same or like parts.

The present disclosure in some embodiments provides a method and apparatus for capacitive force sensing that does not require a rigid or flat boundary or a fixed structural enclosure to operate effectively. The sensor's force transducer can include a flexible conductive outer layer separated from a center conductive layer by one deformable and flexible dielectric material layer where the dielectric stiffness, starting from a base stiffness, increases in proportion to pressure.

The sensing methods of the present disclosure in some embodiments allow for more accurate measuring of total applied force distributed over a fully flexible single-layer or multilayer capacitive transducer. In addition to a new multilayer sensitivity-enhancing vertical interdigitation of conductive and dielectric layers, the present disclosure further teaches the optional use of a dielectric structure having an effective base stiffness that increases in proportion to applied pressure. Using either of these features, individually or in combination, the present disclosure advances the state of the art in total force measurement accuracy over flat or non-flat, dynamically flexing sensing surfaces of the transducer without reliance on rigid surface assumptions or individualized pressure distribution calibration.

By contrast, most conventional force sensors rely either wholly or partially on rigid contact surfaces to translate local pressure into force, which limits their suitability for applications requiring mechanical compliance, low-profile integration, or conformity to curved or flexible substrates. Such rigid-bound sensors are often difficult to embed within soft materials, may be uncomfortable or impractical for wearable use, and can be susceptible to mechanical fatigue, misalignment, or performance degradation under dynamic loading conditions. Existing flexible capacitive transducers designed for force measurement, including systems such as US Patent Publication 2018/0078176 A1 that employ a single flexible dielectric layer, do not achieve the sensitivity, accuracy, or total-force measurement capability provided by the present disclosure without user-specific calibration.

There is therefore a need for force sensors such as provided by the present disclosure that do not depend for functional accuracy on rigid or flat uniform pressure mechanical boundaries, nor are limited in accuracy by the less-than-ideal properties of a single, uniform dielectric layer in the transducer or by the complexities of user-specific calibration, among other advantages described herein. Such sensors are compatible with soft or flexible assemblies, allow for simplified fabrication, and maintain sensitivity, accuracy, and durability in unrestrained configurations. The present disclosure provides for devices that satisfy these needs by introducing a novel capacitive sensing architecture that operates accurately without any rigid boundary constraint, enabling new classes of flexible and unobtrusive force sensing applications.

In an aspect, a flexible capacitive force sensing apparatus can be incorporated into an unobtrusive, comfortable, low-cost sensing insole within a sock, slipper, or shoe that enables total body weight or ground reaction force measurement and tracking while one stands, walks or runs without requiring calibration for a specific individual's plantar pressure pattern. In another aspect, a flexible capacitive force sensing apparatus in a bathroom rug of normal thickness and pliability. The bathroom rug can be capable of measuring body weight. Other aspects include a table mat that can track food consumed from a plate, or a pill box compartment that can sense when a pill has been removed. No comparable solutions exist today for such applications. Conventional force sensors do not have sufficient flexibility, accuracy, or stability.

In some embodiments, conductive and dielectric layers are vertically interdigitated to increase sensor sensitivity and measurement accuracy. Such embodiments enable accurate force measurement at low strain levels using a dielectric whose effective stiffness is approximately constant over the operating strain range. In certain implementations, measurement accuracy is further improved when the dielectric exhibits an effective stiffness that increases as a function of applied pressure. In these embodiments, top and bottom outer electrodes can optionally be held at ground potential to additionally serve as electrical shielding that reduces susceptibility to external electromagnetic interference.

Application of different contact force patterns on the transducer surface causes different localized deformations of the dielectric layers, altering the effective separation between the electrodes differently in different locations. There is no rigid surface to equalize pressure over different regions of the sensing dielectric, nor are there any assumptions of uniform pressure across the surface. Nevertheless, when combined with analog and digital signal processing methods, the transducer can support measurement of the total applied force load.

Unlike traditional capacitive sensors that rely on rigid frames or fixed geometries to maintain alignment and mechanical stability, the disclosed sensor design is inherently compliant and mechanically less constrained. This allows it to conform to curved surfaces, deform with its host structure, and be embedded in soft or flexible substrates without significant degradation in sensing performance. The sensor architecture is further compatible with low-cost fabrication methods and scalable to arbitrary shapes and sizes, making it suitable for a wide range of wearable, biomedical, and robotic applications.

In other embodiments, the device can employ additional conductive and dielectric layers in the transducer that improve sensitivity.

There are also transducer array embodiments with surface encoding that provide a vector of digital signals to estimate a surface distribution and location of capacitance and force. Machine learning methods can process the surface capacitance distribution to estimate total force and force patterns with higher accuracy.

Additional embodiments may include environmental encapsulation or protection components, such as edge or surface sealant to prevent water absorption, or electrical energy harvesting layers responding to pressures.

These and other features discussed herein enable accurate, low-profile, and practical force or motion sensing solutions for use in mechanically dynamic or non-rigid environments. The above and other embodiments will now be discussed with reference to the figures.

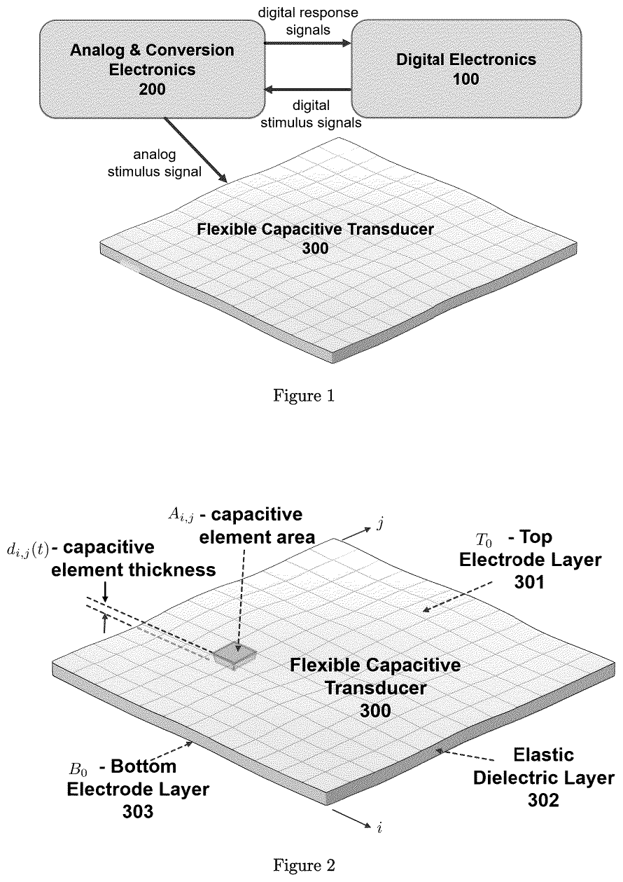

FIG. 1 is a block diagram for the method and apparatus including Digital Electronics (100), Analog & Conversion Electronics (200), and Flexible Capacitive Transducer (300). Digital Electronics (100) can perform digital signal processing that generates digital stimuli and processing digital response signals to measure force. Analog & Conversion Electronics provides analog signal buffering or filtering, and it converts digital signals to analog signals and vice versa.

In one embodiment, the Flexible Capacitive Transducer (300), shown in FIG. 2, includes a flexible conductive Top Electrode Layer (301) that covers the top surface, a flexible and insulating Elastic Dielectric Layer (302), and a flexible conductive Bottom Electrode Layer (303). The transducer layers are laminated to form a deformable capacitor that may cover a large area, but no portion of which needs to be rigid in order to measure total force.

Digital Electronics 100 can generate digital stimuli and collect digital response signals using the analog electronics 200 to convert digital into analog signals and vice versa. Digital signal processing may include machine learning models that use response signals and optionally also stimuli signals to measure capacitance; the measured capacitance can be used to calculate force estimates. The signal processing may be performed locally by an embedded processor or by direct digital implementation such as a field programmable gate array or custom integrated circuit or remotely via a connected device such as a mobile phone. Signal processing methods can include calibration of either linear mappings from capacitance to force or (typically higher accuracy) nonlinear mappings to generate a real-time and continuous stream of force measurement samples. Small, low-power sensor electronics can connect to a thin, flexible, and comfortable transducer 300. The flexible capacitive transducer 300 may have a relaxed state that is not flat, allowing the transducer 300 to be integrated into soft, wearable, or deformable systems. The flexible capacitive transducer 300 can be suitable for integration into environments that need to accurately measure force despite dynamically varying, non-uniform pressure patterns that cause physical flexing and variation of the contact surface geometry. The device of FIG. 1 can allow for accurate and scalable force sensing across non-rigid surfaces using low-cost materials and fabrication processes.

Suitable flexible material options to implement electrode layers, such as Top Electrode Layer (301) or Bottom Electrode Layer (303), include copper or aluminum foils, or conductive cloths, such as silver-coated taffeta or ripstop nylon fabric, copper-coated polyester taffeta fabric, or polyester taffeta with tin over copper. There are many possible elastic materials that are suitable for Elastic Dielectric Layer (302), for example, neoprene or silicon rubber foam sheets.

The top and bottom of Flexible Capacitive Transducer (300) can be divided into a large number of small aligned finite element plate capacitors, where each small plate area A_{i,j} remains large compared to the dielectric thickness d_{i,j}(t) between top and bottom plates, and where each d_{i,j}(t) can vary independently in time from its nominal thickness d_{i,j} due to surface flexibility and varying surface pressure patterns. Nominal d_{i,j} is the resting distance assumed constant when a contact force of interest has a value of 0. It can change if a pervasive background force that is not of interest changes, such as atmospheric pressure.

Such background forces can be assumed to be constant between the start and stop of contact force measurements. Additionally or alternatively, an always unloaded, typically smaller reference capacitor can be added to the system as calibration to compensate for any type of environment change, such as temperature, humidity, or atmospheric pressure.

Further, d_{i,j}(t) can change at each capacitive element position [i,j] on the capacitor surface proportional to the surface pressure, p_{i,j}(t), due to a contact force that may vary across the surface and that in general changes with time t,

$${p}_{i , j} ( t ) = ( {d}_{i , j} - {d}_{i , j} ( t ) ) / {d}_{i , j} ( 0 ) = \text{?} Δ {d}_{i , j} ( t ) / {d}_{i , j} ( 0 ) \qquad ( 1 )$$

Eq. 1 is a first-order model that assumes the dielectric is operating in a region where the coefficient of elasticity, [symbol] , is linear so that change in thickness Δd_{i,j}(t) and change in pressure, p_{i,j}(t), are proportional. p_{i,j}(t) is relative to atmospheric pressure or any other constant background pressure. When p_{i,j}(t)=0, the only pressure is background pressure. d_{i,j}(0) is the starting thickness when surface pressure is due solely to background pressure p_{A}.

This model approximates a flexible transducer when each A_{i,j} is small enough so that the surface is approximately flat over A_{i,j} but each A_{i,j} is large when compared to d_{i,j}(0). This means that all spatial frequencies in the surface with significant energy have periods that are large compared to A_{i,j}. The more curved is the surface containing higher frequencies, the smaller must be A_{i,j} and the thinner must be the dielectric to maintain the approximation. If necessary in practice, various simple and low-cost options can reduce surface undulation frequency, such as covering the transducer with a soft elastic material or a liquid layer that spreads surface forces and avoids spatially impulsive pressure peaks.

Total capacitance Cᵢ(t) approximately equals the sum total capacitance of the small parallel plate capacitors,

$${C}_{i} ( t ) \approx {\sum }_{i , j} \frac{ϵ {A}_{i , j}}{{d}_{i , j} ( t )} = {\sum }_{i , j} \frac{ϵ {A}_{i , j}}{{d}_{i , j} ( 0 ) ( 1 - {δ}_{i , j} ( t ) )} = {\sum }_{i , j} {C}_{i , j} \frac{1}{1 - {δ}_{i , j} ( t )} \qquad ( 2 )$$

where ϵ=ϵᵣϵ₀ is the dielectric permittivity with ϵᵣ being the relative permittivity and ϵ₀=8.854×10⁻¹² F/m being the dielectric permittivity of free space. C_{i,j}=ϵA_{i,j}/d_{i,j}(0) represents the initial unloaded capacitance when Δp_{i,j}(t)=0. δ_{i,j}(t)=Δd_{i,j}(t)/d_{i,j}(0) is the strain of the small parallel plate capacitor at time t.

The overall capacitance change of Flexible Capacitive Transducer (300) from any surface force distribution or pressure pattern can be described by Eq 3,

$$Δ {C}_{i} ( t ) = {C}_{i} ( t ) - {C}_{A} \approx {\sum }_{i , j} \frac{ϵ {A}_{i , j}}{{d}_{i , j} ( t )} - {\sum }_{i , j} \frac{ϵ {A}_{i , j}}{{d}_{i , j} ( 0 )} \approx {\sum }_{i , j} {C}_{i , j} \frac{{δ}_{i , j} ( t )}{1 - {δ}_{i , j} ( t )} \qquad ( 3 )$$

where C_{A} is the unloaded capacitance due to background pressure only,

$${C}_{A} \approx {\sum }_{i , j} {C}_{i , j} \qquad ( 4 )$$

Eq. 3 indicates that if d_{i,j}(0) varies as a function of i and j, then the same pressure at surface points with different d_{i,j}(0) would result in different contributions to the total capacitance change. Because no algorithm that calculates total force from ΔCᵢ(t) alone as input could resolve such an ambiguity, the implication is that base distance d_{i,j}(0) should be a constant d₀ for all of the small, finite capacitive elements.

In this case,

$$Δ {C}_{i} ( t ) \approx \frac{ϵ}{{d}_{0}} {\sum }_{i , j} \frac{{A}_{i , j} {δ}_{i , j} ( t )}{1 - {δ}_{i , j} ( t )} \qquad ( 5 )$$

If the stress/strain curve for compression of the material surface were governed by the following nonlinear stress/strain function:

$${p}_{i , j} ( t ) = \times \frac{{δ}_{i , j} ( t )}{1 - {δ}_{i , j} ( t )} = \text{?} ( {δ}_{i , j} ( t ) + {δ}_{i , j}^{2} ( t ) + {δ}_{i , j}^{3} ( t ) + … ) \qquad ( 6 )$$

where λ is a material stiffness property, then Eq. 5 becomes

$$Δ {C}_{i} ( t ) \approx \frac{ϵ}{λ {d}_{0}} {\sum }_{i , j} {A}_{i , j} {p}_{i , j} ( t ) \qquad ( 7 )$$

Because the total contact force W(t) resulting from any pressure pattern impinging on Flexible Capacitive Transducer (300) is

$$W ( t ) \approx {\sum }_{i , j} {A}_{i , j} {p}_{i , j} ( t ) \qquad ( 8 )$$

$$then$$

$$W ( t ) \approx \frac{λ {d}_{0}}{ϵ} Δ {C}_{i} ( t ) \qquad ( 9 )$$

With the Eq. 6 stress/strain relation, a direct proportionality between capacitance and force holds in principle for any level of strain.

The next step is to find or engineer an elastic material that exhibits the desired ideal stress/strain curve of Eq. 6. For small strains, e.g. δ_{i,j}(t)<0.02, this curve is approximately linear,

$${p}_{i , j} ( t ) = {λδ}_{i , j} ( t ) \qquad ( 10 )$$

It is well-known that a linear model is a good approximation for almost all materials under a small strain assumption, which in this case means that there are many suitable elastic material options with no special engineering required.

Measurement sensitivity is proportional to ΔCᵢ(t) which is proportional to transducer base capacitance and inversely proportional to stiffness coefficient, λ, that also sets the force measurement range and accuracy. Larger λ results in smaller strain for the same pressure. This implies better force measurement accuracy at the cost of higher resolution measurement of smaller capacitance changes. Implementation must find a sweet spot in this trade-off. However, the multilayer transducer designs described below significantly amplify sensitivity, and thus better support large λ with small strain.

One approach to approximating Eq. 6 more closely than does a small strain linear model is to introduce a surface texture into the dielectric. Examples include an array of geometric unit cells such as dimples (e.g., square or hemispherical) or a honeycomb mesh (such as hollow walled triangles or squares or hexagons). In addition to a surface texture, a mesh geometry may also constitute the entire dielectric or form a distinct mid-plane layer. As discussed herein, such a texture, which may include gaps, can permit the use of volume-incompressible elastomers in the dielectric. The texture can enable one-dimensional compression to be governed primarily by the elastic response of the pillars (the region defined between the gaps) rather than by the high bulk modulus of a nearly incompressible volume

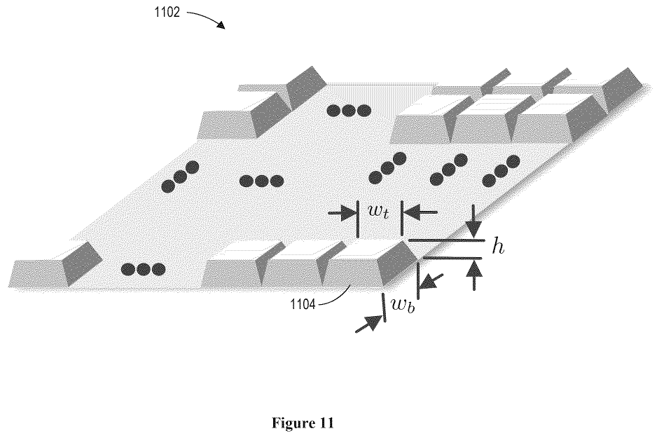

One example texture is an array of repeating square, flat-topped pyramid unit cells (also referred to as pillars) as shown in FIG. 11. Each cell 1104 of the array 1102 has sloped walls with bottom edge width, w_{b}, top edge width wₜ<w_{b}, and height h. The pitch or cell center to center spacing of this particular example is also w_{b}. Assume Poisson's ratio for the elastic material is small so that bulk constraints can be ignored. An example is Rogers HT-840 silicone elastic foam.

Each pyramid in the array occupies a cell area of

$${A}_{cell} = {w}_{b}^{2} .$$

When a force is applied to the transducer, the force per unit cell is

$${F}_{i , j} ( t ) = {p}_{i , j} ( t ) {w}_{b}^{2} \qquad ( 10 )$$

where p_{i,j}(t) is the force per unit cell area. However, pressure on the pyramid top is

$${p}_{i , j}^{*} ( t ) = {p}_{i , j} ( t ) \frac{{w}_{b}^{2}}{{( {w}_{t} + 2 Δ {w}_{i , j} ( t ) )}^{2}} \qquad ( 11 )$$

where 2Δw_{i,j}(t) is the change in the width of the contact area square when increasing compressive pressure causes a change in height, Δh_{i,j}(t).

If the material stress is linearly proportional to strain (small to moderate strain for an elastic foam),

$${p}_{i , j} ( t ) \frac{{w}_{b}^{2}}{{( {w}_{t} + 2 Δ {w}_{i , j} ( t ) )}^{2}} = {E}_{0} \frac{Δ {h}_{i , j} ( t )}{h} = {E}_{0} {δ}_{i , j} ( t ) \qquad ( 12 )$$

Similar triangles in the pyramid cross-section indicate that Δw_{i,j}(t) relates to Δh_{i,j}(t) as follows:

$$\frac{Δ {h}_{i , j} ( t )}{Δ {w}_{i , j} ( t )} = \frac{h}{( {w}_{b} - {w}_{t} ) / 2} \qquad ( 13 )$$

$$Δ {w}_{i , j} ( t ) = \frac{Δ {h}_{i , j} ( t )}{h} \frac{{w}_{b} - {w}_{t}}{2} = {δ}_{i , j} ( t ) \frac{{w}_{b} - {w}_{t}}{2} \qquad ( 14 )$$

Substitute for Δw_{i,j}(t) into Eq. 12 and simplify,

$${p}_{i , j} ( t ) = {E}_{0} {δ}_{i , j} ( t ) ( \frac{{w}_{t}^{2}}{{w}_{b}^{2}} ) {( 1 + {δ}_{i , j} ( t ) ( \frac{{w}_{b} - {w}_{t}}{{w}_{t}} ) )}^{2} = {E}_{0} ( \frac{{w}_{t}^{2}}{{w}_{b}^{2}} ) ( {δ}_{i , j} ( t ) + 2 ( \frac{{w}_{b} - {w}_{t}}{{w}_{t}} ) {δ}_{i , j}^{2} ( t ) + {( \frac{{w}_{b} - {w}_{t}}{{w}_{t}} )}^{2} {δ}_{i , j}^{3} ( t ) ) \qquad ( 15 )$$

If we select w_{b} and wₜ so that

$$\frac{{w}_{b} - {w}_{t}}{{w}_{t}} = \frac{1}{2} \qquad ( 16 )$$

$$then$$

$${p}_{i , j} ( t ) = {E}_{0} ( \frac{{w}_{t}^{2}}{{w}_{b}^{2}} ) ( {δ}_{i , j} ( t ) + {δ}_{i , j}^{2} ( t ) + {δ}_{i , j}^{3} ( t ) / 4 ) \qquad ( 17 )$$

This is a second order approximation to the ideal stress/strain curve of Eq. 6 that supports an order of magnitude larger strain and hence higher sensitivity than a first order linear approximation while maintaining the same accuracy. For Rogers HT-840 silicone foam, one possible set of suitable parameter values are w_{b}=2 mm, wₜ=1 mm, and h=0.87 mm.

To measure total force on the transducer, it is necessary to measure total change in capacitance. Accuracy and resolution of force measurement depend on the accuracy and resolution of the capacitance change measurement. Capacitance measurement can be done using a well-known and suitable Digital Electronics (100) implementation, such as a microcontroller, digital signal processor, field programmable gate array, or integrated circuit. Digital signal processing operates in conjunction with a corresponding Analog & Conversion Electronics (200) that supports capacitance measurement. Such Analog & Conversion Electronics (200) may be known in the art.

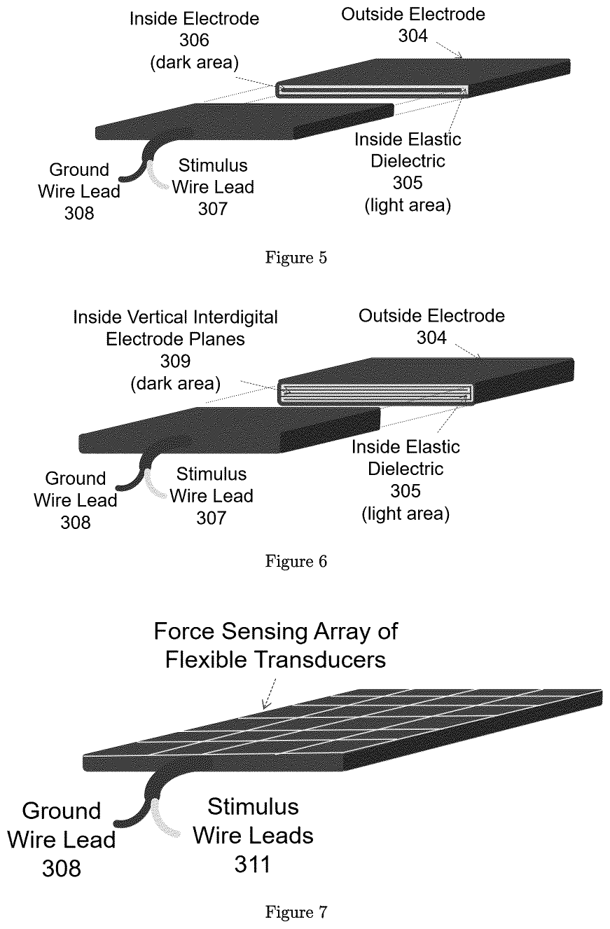

FIG. 5 shows an alternative embodiment of Flexible Capacitive Transducer (300) that provides broadband shielding from powerline and other noise interferences as well as enhancing sensitivity. This embodiment includes Outside Electrode (304) surrounding Inside Dielectric (305) as well as Inside Electrode (306). Outside Electrode (304) would typically be tied to the ground reference, while Analog & Conversion Electronics (200) would deliver an analog stimulus to Inside Electrode (306) via Stimulus Wire Lead (307). For further improved interference shielding, a ground shield would also wrap the insulation of Stimulus Wire Lead (307).

The transducer in FIG. 5 can include two dielectric layers. Each layer can include area A and dielectric permittivity E. It can be assumed that the relaxed thickness for each dielectric layer is d₀/2 so that total thickness is the same as the single dielectric layer transducer. Because the dielectric layers are parallel capacitors, a component in the finite element model of this capacitor is

$${C}_{i , j}^{2} = \frac{ϵ {A}_{i , j}}{{d}_{0} / 2} + \frac{ε {A}_{i , j}}{{d}_{0} / 2} = \frac{4 {ϵA}_{i , j}}{{d}_{0}} = 4 {C}_{i , j}^{1} \qquad ( 17 )$$

where the superscript 2 indicates two dielectric layers.

Eq. 17 implies that capacitance goes up by a factor of 4. Because Eq. 7 shows that capacitance change or sensitivity is proportional to capacitance, sensitivity also improves by a factor of 4. This is a significant advantage because it implies that λ can be 4 times that of the single dielectric layer transducer for the same total transducer dielectric thickness and range of force measurement. Because a larger [symbol] implies a stiffer, less compressible material, the strain in each layer is ¼ of that of a single layer transducer, while each layer thickness is ½ of a single layer. The strain in each layer can therefore be ½ of that of a single layer, which means that each layer also better satisfies condition Eq. 6.

Along similar lines, N layers that create N parallel capacitors can be described by Eq. 18.

$${C}_{i , j}^{N} = N \frac{ϵ {A}_{i , j}}{{d}_{0} / N} = \frac{{N}^{2} ϵ {A}_{i , j}}{{d}_{0}} = {N}^{2} {C}_{i , j}^{1} \qquad ( 18 )$$

Capacitive measurement sensitivity goes up by a factor of N², implying that λ can be N² times that of the single dielectric layer transducer for the same range of force measurement. For the same total sensor thickness, this implies that the strain in each layer is 1/N of that of a single layer, meaning that an N-layer transducer can better meet the condition given by Eq. 6 as N grows larger.

FIG. 6 illustrates an embodiment for an N-layer Flexible Capacitive Transducer (300) with N=6 dielectric layers in this case, where Outside Electrode (304) is typically tied to the system ground reference and provides broadband shielding from powerline and other noise interferences. Outside Electrode (304) surrounds Inside Dielectric (305) as well as Inside Electrodes (309). Inside Electrodes (309) alternate between signal and ground electrodes. Outside Electrode (304) would typically be tied to the ground reference, while Analog & Conversion Electronics (200) would deliver the same analog stimulus to each signal electrode of Inside Electrodes (309) via Stimulus Wire Lead (307). For further improved interference shielding, a ground shield would also wrap the insulation of Stimulus Wire Lead (307).

In order to measure force distribution across the surface, as well as total force more accurately by adding more cost and complexity, N-layer flexible transducers can be concatenated into a larger M×P sensor array as shown in FIG. 7, where M, N, and P are integers greater than or equal to 1. Each array element is a completely separate force transducer receiving one stimulus from Stimulus Wire Leads (311). However, ground electrode layers or the dielectric layers of these transducers can be in direct contact, or they need not be subdivided into array elements at all. Only non-grounded conductive electrodes that are driven by a stimulus signal need be subdivided into separate array elements. For further improved interference shielding, a ground shield would also wrap the insulation of Stimulus Wire Leads (311). This architecture requires M×P signal wire leads plus 1 ground wire lead.

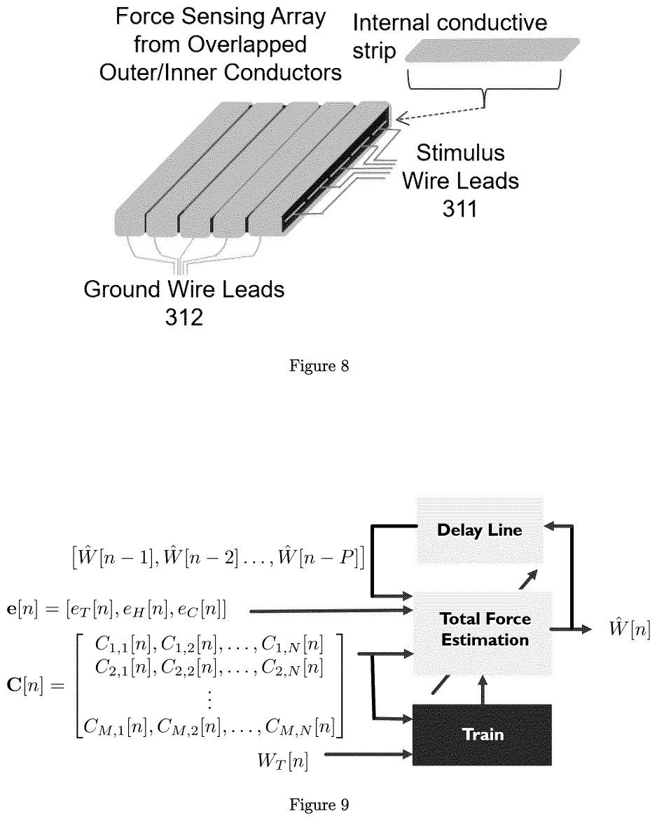

An option both to reduce the number of required wire leads and support connections to the outer edge only of the M×P sensor array is to form the array of capacitive transducer elements from overlapping ground and signal conductive strips as shown in FIG. 8. This design requires M outer conductor leads plus P inner conductor leads that encode each array element. Ground applied to one of the coordinates i=0, . . . . M−1 with signal applied to one of the coordinates j=0, . . . . P−1, for example, selects the (i, j) array element.

Calibration signal processing illustrated in FIG. 8 for the single capacitor or the array architecture illustrated in FIG. 8 or 9 can improve measurement by compensating for transducer nonlinearity, nonuniformity, environmental conditions such as temperature and humidity, or material wear and tear that otherwise would result in decay of measurement accuracy. Because Eq. 9 makes several idealizations, it is believed that the accuracy of mapping the capacitance matrix of the entire array ΔCᵢ[n] to force Wᵢ[n] can improve by using an empirically calibrated mapping that accounts for nonlinear material properties, environment, and other nonidealities.

$$\hat{W} [ n ] = F ( \hat{W} [ n - 1 ] , … , \hat{W} [ n - M ] , C [ n ] , … , C [ n - N ] , e [ n ] , … , e [ n - P ] ) \qquad ( 14 )$$

F(⋅) estimates the current force from past values of its outputs and from the current and past values of its inputs that in addition to the array of capacitances, C[n], includes a vector of environmental measurements, e[n], such as temperature e_{T}[n] or humidity e_{H}[n] or a reference capacitance e_{C}[n].

One option to find a suitable F(⋅) uses machine learning with known correct values W_{T}[n] to train a mapping model as illustrated in FIG. 9. Note that the smallest array for this signal processing is a single capacitor transducer, M=N=1.

Any embodiments may include various other features, including, for example, any features described in any of the following references: CA 2352768 A1, discussing a capacitive force sensor with feedback circuit and rigid housing; U.S. Pat. No. 7,395,717, discussing a flexible capacitive sensor with external signal interpretation; US 2018/0078176, discussing a force sensor; US 2020/0064918, discussing a capacitive flex sensor on wearable or bendable substrates; Muzaffar et al. “Shoe Integrated, Force Sensor Design for Continuous Body Weight Monitoring,” Italian National Conference on Sensors, June 2020; Mannsfeld et al., “Highly sensitive flexible pressure sensors with microstructured rubber dielectric layers,” Nature Materials 9, pages 859-864 (2010); Kwon et al., “Porous Dielectric Elastomer Based Ultra-Sensitive Capacitive Pressure Sensor and Its Application to Wearable Sensing,” Proceedings of the 18th International Conference on Solid-State Sensors, Actuators and Microsystems (TRANSDUCERS); pages 604-607 (2015); and Gerratt et al., “Elastomeric Electronic Skin for Prosthetic Tactile Sensation,” Advanced Functional Materials; volume 25, number 15, pages 2287-2295 (2015); each of which is incorporated by reference in its entirety and forms a part of this specification for all purposes.

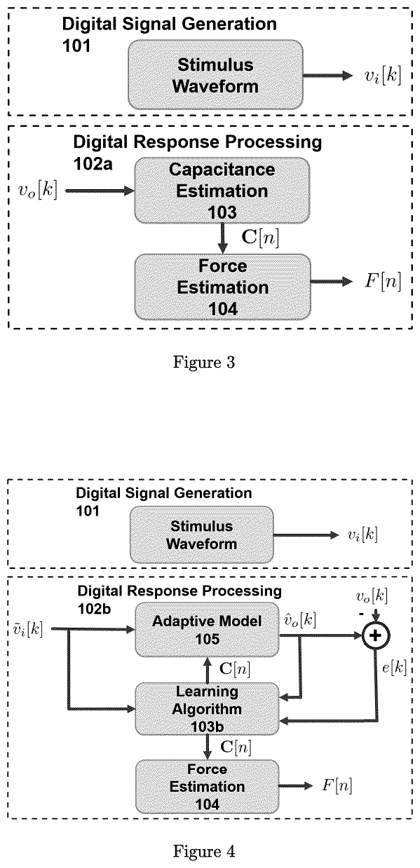

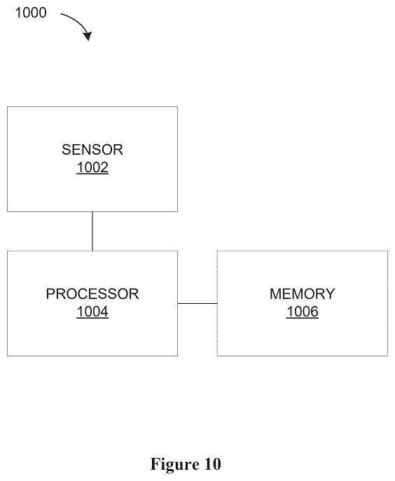

In an aspect, the present disclosure provides for a computing system 1000 as shown in FIG. 10. Various methods in accordance with the present disclosure can be implemented on computing system 1000. Computing system 1000 can include a sensor 1002, a processor 1004, and a memory 1006. In some examples, one or more of the sensor 1002, the processor 1004, and/or the memory 1006 can be integrated into a single device. In some examples, the sensor 1002 may be in electrical communication with the processor 1004. In some examples, the sensor 1002 and the processor 1004 may be in wireless communication. In some examples, the processor 1004 and/or the memory 1006 may be cloud-based, and the sensor 1002 may be capable of communicating with the cloud-based network. The sensor 1002 may include any of the sensor components discussed herein, for example with reference to FIG. 1-2 or 5-8. The memory 1006 may include instructions for the processor 1004 to execute any of the processes discussed herein, for example any of the processes discussed herein with reference to FIG. 3, 4, or 9. It is to be understood that the computing system 1000 may include one or more of each of the sensor 1002, the processor 1004, and/or the memory 1006. The computing system 1000 may be able to communicate with a user device, for example a personal computing device or a mobile device. In some examples, the computing system 1000 optionally includes a display, which can be used to display parameters calculated from measurements of the sensor 1002.

In some embodiments, software written to perform the methods as described herein is stored in memory 1006 in some form of computer readable medium, for example memory, CD-ROM, DVD-ROM, memory stick, flash drive, hard drive, SSD hard drive, server, mainframe storage system and the like.

In some embodiments, the methods of the processor 1004 or the memory 1006 may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A capacitive force sensing apparatus comprising:
(a) a flexible capacitive transducer, the flexible capacitive transducer comprising electrodes separated by at least one deformable dielectric, the flexible capacitive transducer defining a pressure-integrating sensing surface;
(b) a capacitance measurement circuit coupled to the electrodes and configured to measure an aggregate capacitance of the flexible capacitive transducer; and
(c) one or more processors configured to execute instructions to:
(i) estimate the aggregate capacitance indicative of a spatially non-uniform pressure distribution applied across the pressure-integrating sensing surface, and
(ii) determine, from the aggregate capacitance, a quantitative estimate of a total applied force,
wherein:
the quantitative estimate of total applied force is substantially invariant to a spatial distribution of the applied pressure over the sensing surface, and
the capacitive force sensing apparatus is without a rigid backing plate, a rigid load-spreading structure, or a rigid boundary that enforces a uniform pressure field across a surface of the flexible capacitive transducer.

2. The apparatus of claim 1, wherein the flexible capacitive transducer comprises a plurality of flexible electrode layers and a plurality of deformable dielectric layers vertically interdigitated to form a multilayer electrode-dielectric stack defining a plurality of capacitors electrically connectable in parallel.

3. The apparatus of claim 2, wherein the flexible capacitive transducer forms a multilayer electrode-dielectric stack having an aggregate capacitance that increases approximately with a square of a number of dielectric layers in the stack.

4. The apparatus of claim 2, wherein the multilayer electrode-dielectric stack comprises alternating signal electrode layers and reference electrode layers.

5. The apparatus of claim 4, wherein at least one reference electrode layer is held at a ground or guard potential, wherein the at least one reference electrode layer held at a ground or guard potential is configured to provide electromagnetic interference shielding.

6. The apparatus of claim 2, wherein the multilayer electrode-dielectric stack comprises at least three dielectric layers.

7. (canceled)

8. The apparatus of claim 1, wherein an effective compressive stiffness of the at least one deformable dielectric approximates:
$$p = {E}_{0} \frac{δ}{1 - δ}$$
for pressure p and strain δ over an operating pressure range.

9. The apparatus of claim 1, wherein an effective compressive stiffness of the at least one deformable dielectric increases with applied pressure, and the increase enables an approximately proportional relationship between capacitance change and total applied force over a larger strain range than a constant-stiffness dielectric.

10. The apparatus of claim 1, wherein the at least one deformable dielectric comprises a structured or composite dielectric configured to produce an effective stiffness increase with applied pressure.

11. The apparatus of claim 1, wherein:
the flexible capacitive transducer comprises a multilayer electrode-dielectric stack with vertically interdigitated electrode and dielectric layers forming a plurality of capacitors in parallel; and
at least one dielectric layer of the multilayer electrode-dielectric stack exhibits an effective compressive stiffness that increases with applied pressure.

12. (canceled)

13. The apparatus of claim 1, wherein the one or more processors is further configured to execute instructions to apply a calibrated mapping from measured capacitance to total force, wherein the calibrated mapping is nonlinear and compensates for at least one of dielectric nonlinearity, environmental variation, or material aging.

14. The apparatus of claim 1, wherein the one or more processors is further configured to execute instructions to apply a calibrated mapping from measured capacitance to total force, wherein the calibrated mapping is trained using supervised learning with reference force values.

15. (canceled)

16. (canceled)

17. (canceled)

18. The apparatus of claim 1, wherein the flexible capacitive transducer conforms to a non-planar surface in an unloaded state.

19. The apparatus of claim 1, wherein the flexible capacitive transducer is integrated into a wearable or deformable structure selected from an insole, shoe, slipper, sock, textile, rug, mat, or robotic contact surface.

20. A flexible capacitive transducer comprising:
(a) flexible electrode layers; and
(b) at least one deformable dielectric separating opposing electrode layers; wherein the flexible capacitive transducer is configured to produce an aggregate capacitance change representative of a total force applied under non-uniform pressure distributions without requiring a rigid backing structure.

21. The flexible capacitive transducer of claim 20, wherein the flexible capacitive transducer comprises vertically interdigitated electrode and dielectric layers forming a plurality of capacitors in parallel.

22. The flexible capacitive transducer of claim 20, wherein the at least one deformable dielectric exhibits an effective compressive stiffness that increases with applied pressure according to
$$p = {E}_{0} \frac{δ}{1 - δ} .$$

23. The flexible capacitive transducer of claim 20, wherein the flexible capacitive transducer includes both vertically interdigitated electrode-dielectric layers and a pressure-dependent effective dielectric stiffness.

24. A method for sensing force comprising:
applying a non-uniform pressure distribution to a flexible capacitive transducer measuring a capacitance-dependent signal from the flexible capacitive transducer in response to the non-uniform pressure distribution being applied to the flexible capacitive transducer, the flexible capacitive transducer comprising flexible electrodes separated by at least one deformable dielectric;
estimating capacitance from the capacitance-dependent signal; and
generating an estimate of a total applied force from the estimated capacitance, wherein the method is performed without constraining the flexible capacitive transducer to a rigid planar boundary.

25. The method of claim 24, wherein the flexible capacitive transducer comprises vertically interdigitated electrode and dielectric layers forming a plurality of capacitors in parallel.

26. The method of claim 24, further comprising increasing an effective compressive stiffness of the at least one deformable dielectric in response to increasing the applied non-uniform pressure distribution.

27. (canceled)
